(19) United States
(12) Patent Application Publication
(10) Pub. No.: US 2022/0010263 A1
(43) Pub. Date: Jan. 13, 2022

(54) SUPERFLUIDS DISRUPTION OF SACCHAROMYCES CEREVISIAE (YEAST), CELL WALL DISINTEGRATION INTO NANOPARTICLES AND FRACTIONATION INTO BETA-GLUCANS, CHITIN AND MANNANS (MANNOPROTEINS)

(71) Applicant: Trevor Percival Castor, Arlington, MA (US)

(72) Inventor: Trevor Percival Castor, Arlington, MA (US)

(73) Assignee: Aphios Corporation

(21) Appl. No.: 17/374,966

(22) Filed: Jul. 13, 2021

Related U.S. Application Data
(60) Provisional application No. 63/051,079, filed on Jul. 13, 2020.

(51) Int. Cl. C12N 1/06; C12P 19/04

(52) U.S. Cl. CPC C12N 1/066; C12P 19/04; C12N 1/063

(57) ABSTRACT

The present invention is directed to methods and apparatus for and products from disrupting, removing intracellular proteins, enzymes and nucleic acids, spray drying, lipid extraction, and making nanoparticles of Saccharomyces cerevisiae (yeast) cell wall followed by acid and/or enzymatic hydrolysis to produce Beta (β)-glucans, chitin and mannans (mannoproteins). The process and apparatus feature critical, supercritical, or near critical fluids for disruption of yeast and making yeast cell wall nanoparticles. The product materials retain full activity and are devoid of residual processing chemicals such as solvents, salts, or surfactants.

### REFERENCES TO OTHER PATENTS

This nonprovisional patent application claims priority to U.S. provisional application Ser. No. 63,051,079 filed on Jul. 13, 2020, the contents of which is incorporated by reference in its entirety.

### FIELD OF INVENTION

The present invention is directed to methods and apparatus for, and products from disrupting, spray drying, extracting and hydrolyzing Saccharomyces cerevisiae (yeast) to produce Beta (β)-glucans, chitin and mannans (mannoproteins). The process and apparatus feature critical, supercritical, or near critical fluids with or without cosolvents for disruption of yeast, removal of intracellular proteins, enzymes and nucleic acids, extraction of lipids and making yeast cell wall nanoparticles.

This application discloses a number of improvements and enhancements to supercritical fluid disruption and extraction from microbial cells disclosed in U.S. Pat. No. 5,380,826 by Castor et al. (1995), which is hereby incorporated by reference in its entirety.

This application discloses a number of improvements and enhancements to method for size reduction of proteins and apparatus disclosed in U.S. Pat. No. 6,051,694, Castor et al. (2000), which is hereby incorporated by reference in its entirety.

### BACKGROUND OF THE INVENTION

Yeast cell walls consist of 70% neutral carbohydrate (polysaccharides), 7% amino sugars, 15% lipids and 0.8% phosphorous (Vega et al., 1986). The three main polysaccharide groups are β-glucans, polymers of mannose (mannoproteins known as mannans), around 60%. 40% and 2% respectively.

These polysaccharides are finding wide benefits in food, pet-food and feed products as well as dietary supplements. Insoluble β-glucans are reported to have immune modulation effects against infectious disease and cancer and enhanced antibiotic efficiency on infections with antibiotic resistant bacteria. β-glucans from Bakers' yeast have received GRAS status from the FDA in 1997 and are regulated in Europe as a “novel food.” It has also been shown that mannan improves gastrointestinal health by preventing of pathogens to host's cells.

### SUMMARY OF THE INVENTION

In this invention, SuperFluids carbon dioxide can be used to disrupt Saccharomyces cerevisiae (Yeast) per “Supercritical Fluid Disruption and Extraction from Microbial Cells.” U.S. Pat. No. 5,380,826 by Castor et al. (1995). SuperFluids are supercritical fluids, critical fluids and/or near-critical fluids with or without polar cosolvents. This '826 patent is incorporated in full by reference in this disclosure.

Yeast in a slurry is first saturated with SuperFluids CO₂ at operating pressures between 2,000 and 5,000 psig and temperatures between 20 and 60° C. After saturation, the yeast slurry is rapidly decompressed into a decompression chamber. As a result of expansive forces, yeast is disrupted and intracellular proteins, enzymes and nucleic acids are released and can be recovered.

In an embodiment of this invention, during decompression, the yeast solution can be heated so the liquid solvent (water) evaporates, and the disrupted yeast is dried into a powder as in a spray drier. In another embodiment, yeast can also be decompressed into a fully or partially evacuated chamber to achieve a spray drying effect. A combination of heat and low pressure can be utilized to produce a spray-dried disrupted yeast powder.

In another embodiment of this invention, SuperFluids CO₂ at appropriate conditions of temperature and pressure can then be used to extract and remove lipids from the spay-dried yeast powder. Spray dried disrupted yeast powder is contacted with SuperFluids CO₂ at operating pressures between 2,000 and 20,000 psig and temperatures between 20 and 100° C. to solubilize and remove lipids.

In another embodiment of this invention, the lipid-reduced, disrupted, spray-dried yeast powder saturated with SuperFluids C0₂ is rapidly expanded to produce yeast wall nanoparticles. This process is similar to “Method for Size Reduction of Proteins,” U.S. Pat. No. 6,051,694, Castor et al. (2000). This '694 patent is incorporated in full by reference in this disclosure.

In another embodiment of this invention, the lipid-reduced, disrupted, spray-dried yeast nanoparticles are processed by enzymatic cleavage to produce fractions of β-glucans, chitins and mannans.

In another embodiment of this invention, the lipid-reduced, disrupted, spray-dried yeast nanoparticles are processed by hydrolysis to produce fractions of β-glucans, chitins and mannans.

In another embodiment of this invention, the lipid-reduced, disrupted, spray-dried yeast nanoparticles are processed by a combination of acid hydrolysis and enzymatic cleavage to produce fractions of β-glucans, chitins and mannans.

These and other features of the invention are described in greater detail below.

### BRIEF DESCRIPTION OF THE DRAWINGS

FIG. 1 illustrates the yeast fractionation process of the present invention;

FIG. 2 schematically illustrates an apparatus capable of continuous yeast cell disruption; and

FIG. 3 schematically illustrates an apparatus for performing the lipid extraction of the present invention;

FIG. 4 schematically illustrates an apparatus for making yeast cell nanoparticles.

### DETAILED DESCRIPTION

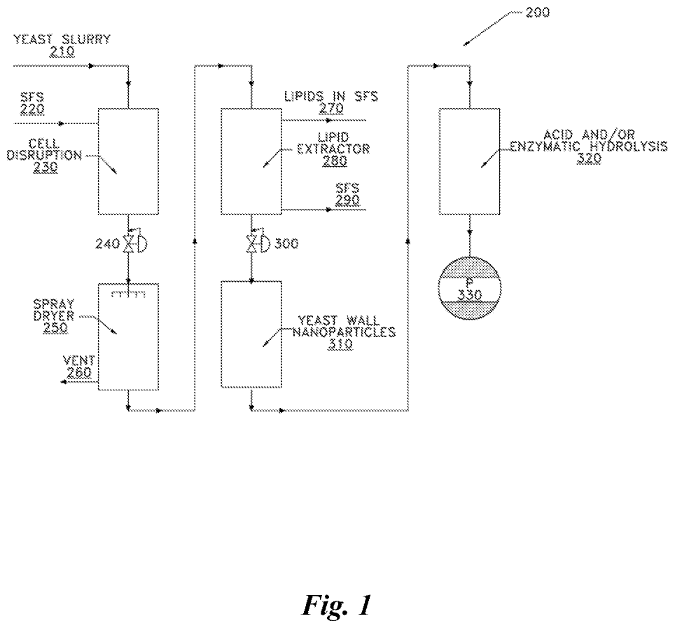

FIG. 1 illustrates the basic process 200 of yeast fractionation according to the present invention. The object of this fractionation process is to disrupt yeast, remove intracellular proteins and enzymes, extract lipids from the yeast cell walls, make yeast cell wall nanoparticles and use acid and/or enzymatic hydrolysis to produce fractions of β-glucans, chitins and mannans.

Yeast in the form of slurry 210 is introduced to a SuperFluids (SFS) chamber at specified temperature and pressure sufficient for the SFS 220 to penetrate the cell walls of the yeast and saturate the yeast with SFS in 230. SFS used includes carbon dioxide, nitrous oxide, propane, alkanes and fluorocarbons. A preferred SFS is carbon dioxide. Pressures range from 500 psig to 5,000 psig. A preferred pressure is 3,000 psig. Temperatures range from 10° C. to 60° C. A preferred temperature is 40° C.

The SFS saturated yeast is rapidly decompressed via a back-pressure regulator 240 through a high-pressure single fluid nozzle (500 psig to 5,000 psig) into chamber 250 which also acts as spray drier that is exhausted by vent 260 which can be connected to a vacuum source. As a result of decompression, yeast is disrupted releasing intracellular proteins, enzymes and nucleic acids. The disrupted yeast is spray dried as a result of low pressure (0 psia to 200 psia) and higher temperatures in the spray drier (60° C. to 200° C.).

The spray-dried disrupted yeast cells in a powdered form are then contacted with SuperFluids (SFS) 290 in an extractor 280 to remove lipids in SFS 270. SFS used includes carbon dioxide, nitrous oxide, propane, alkanes and fluorocarbons. A preferred SFS is carbon dioxide. Pressures range from 500 psig to 5,000 psig. A preferred pressure is 3,000 psig. Temperatures range from 10° C. to 60° C. A preferred temperature is 40° C.

The delipidated, spray-dried disrupted yeast cells in a powdered form are then expanded through decompression valve 300. Rapid expansion of the SFS causes explosive disruption of the yeast cell walls producing yeast cell wall nanoparticles 310.

As a final process step 320, enzymatic cleavage of yeast cell nanoparticles produces Beta-glucans, chitins and mannans fractions. Hydrolysis, which may be acid-based, may also be performed on the cell nanoparticles to produce Beta-glucans, chitins, and mannans fractions. The fractionated yeast products 330 are produced in the final step of the process. The final process step can consist of a combination of enzymatic hydrolysis and acid hydrolysis.

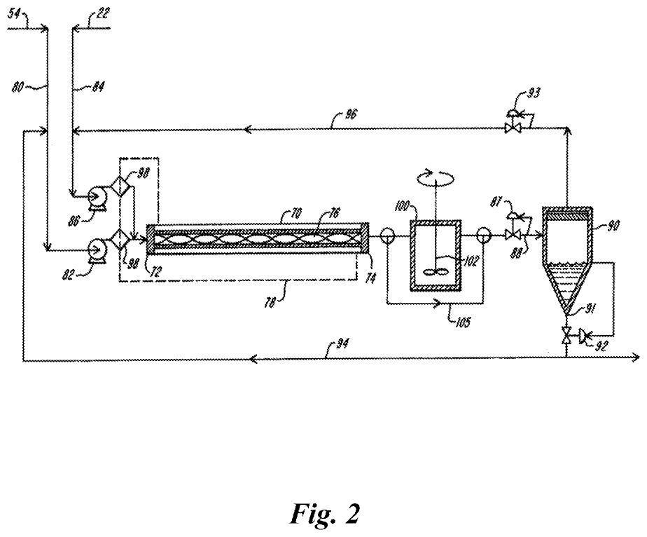

The apparatus of shown in FIG. 2 is designed for continuous SuperFluids disruption of yeast. The apparatus includes a mixing chamber 70 in the form of an elongated cylinder having an inlet end 72 and outlet end 74. Disposed centrally throughout the mixing chamber 70 is a static mixer 76. The static mixer 76 mixes the yeast slurry 54 and the solvent 22 as the mixture is directed continuously from the inlet end 72 to the outlet end 74 of the mixing chamber 70. The mixing chamber 70 is jacketed and interfaced with a temperature control loop 78 which recovers the heat of compression of the solvent as well as any heat transferred from fermenters and centrifuges. The temperature control loop 78, of course, is capable of maintaining the contents of the mixing chamber 70 at a preset temperature.

A slurry conduit 80 for introducing a slurry of yeast cells into the mixing chamber 70 communicates with the inlet end 72. A high-pressure slurry pump 82 is connected to the slurry conduit 80 for pumping the slurry of cells under pressure into the mixing chamber 70. A solvent conduit 84 is in fluid communication with the slurry conduit 80 downstream of the slurry pump 82. A compressor 86 is provided along the solvent conduit 84 for raising the pressure of the solvent 22 and of the mixture within the mixing chamber 70 to critical pressures and above. A discharge conduit 88 leads from the outlet end 74 of the mixing chamber 70 to a blow-down chamber 90. A back pressure regulator or valve 87 is placed along the discharge conduit 88 between the mixing chamber 70 and the blow-down chamber 90 for continuously releasing the pressure on the slurry of cells exiting from the mixing chamber 70.

The blow-down chamber 90 is constructed and arranged to allow effective gravity separation of the solvent and the disrupted yeast slurry. In the embodiment shown, the lower end of the blow-down chamber 90 is funnel-shaped for collecting the disrupted cells. At the bottom of the funnel is an exit port 91. A liquid level control valve 92 is attached at the bottom exit port of the blow-down chamber 90 for controlling the liquid level within the blow-down chamber. Material may be collected at this port 91 or recycled via slurry recycle conduit 94 to the slurry conduit 80 upstream of the slurry pump 82.

A solvent recycle conduit 96 fluidly connects the upper exit of the blow-down chamber 90 to the solvent conduit 84, upstream of the compressor 86. Another back-pressure regulator 93 is located on the solvent recycle conduit 96 for controlling the pressure within the blow-down chamber 90.

Heat exchangers 98 are located just downstream of the solvent compressor 86 and the slurry pump 82 to regulate the temperature of solvent leaving the compressor. The temperature control loop 78 also controls the heat exchangers 98.

In operation, yeast cell slurry 54 may be fed directly from fermenters or centrifuges into the apparatus of FIG. 2. The slurry 54 is pumped with the high-pressure slurry pump 82 into the mixing chamber 70. Recycled solvent and any necessary make-up solvent are compressed and added to the yeast cell slurry downstream of the slurry pump 82 and upstream of the mixing chamber 70. The mixture of cells and solvent then is introduced continuously into the mixing chamber 70 and the mixture passes from the inlet end to the outlet end while being continuously mixed. The mixture continuously exits from the mixing chamber 70. As it exits, it is rapidly expanded through the heated, pressure-reduction valve 87 and is tangentially ejected into the blow-down chamber 90. Once in the blow-down chamber 90, the disrupted yeast slurry settles to the bottom and the solvent 22 stays on top. The separated solvent then may be recycled and used again. The disrupted slurry may be collected or may be recycled to increase the average residence time through the mixing chamber 70.

The pressure of the blow-down chamber may be maintained at pressures ranging from atmospheric to that of the mixing chamber. For a dominant coloration or permeability improvement mechanism, the pressure in the blow-down chamber 90 may be maintained at pressures relatively close to the operating pressures of the mixing chamber 70.

The continuous flow apparatus also may include a soaking chamber between the mixing chamber 70 and the blow-down chamber 90. Such a soaking chamber 100 will allow for a longer exposure time between the SFS solvent and the yeast cells; the soaking chamber may also accommodate mechanical mixers 102 to further facilitate the saturation of each yeast cell with SFS solvent. The soaking chamber can be bypassed by allowing the mixture of supercritical fluid and yeast slurry to flow directly from the mixing chamber 70 to blowdown chamber 90 via bypass loop 105.

For the process scribe in connection with FIG. 2, Saccharomyces cerevisiae Baker's yeast was aerobically grown in a fed-batch mode with glucose as the only limiting nutrient at a temperature of 30° C. and a pH of 5.0. Dissolved gas was kept above 15% through appropriate increase of air flow and agitation. Glucose was fed continuously; the glucose flow was determined by a computer control strategy which avoids ethanol production and keeps the specific growth rate around 0.22 l/hr. Ammonia was used as the nitrogen source and was fed as needed vis-a-vis a pH controller. The final cell density reached at the moment of harvesting was 51 grams dry cell weight per liter (g DCW/l). The cells were harvested after cooling down the fermenter to 22 degrees centigrade, whereupon 3,200 ml of broth was collected for gravity sedimentation. After 48 hours, the supernatant was withdrawn and 1,200 ml of concentrated suspension was collected. The concentrated suspension contained approximately 136.0 g DCW/l since gravity sedimentation concentrated the suspension by a factor of 2.67.

The effect of pressure on the supercritical disruption of Baker's yeast using N₂O was also tested. The temperature and recirculation time were fixed at 40° C. and 25 minutes respectively. Pressure was varied from about 1,100 psig to 4,800 psig. As pressure increased, the recovery of nucleic acids and protein also increased. However, the relationship was more linear than that for E. coli, indicating that higher pressures may result in even higher recovery efficiencies.

The present invention utilizes SuperFluids to fractionate cellular biomass materials in two steps. In the first step, the biomass is disrupted by exposure to the critical fluid. It is hypothesized that this disruption involves at least two mechanisms, the first being liberation of cell envelope constituents to cause cell envelope permeability. The cell envelope constituents are not necessarily solvated in the critical fluid, i.e., they may remain in the phase containing the biomass, but in any case lose their structural association with the cell. The resulting permeability of the cell envelope makes certain contents of the cell accessible to be extracted in subsequent steps.

The second mechanism of disruption involves an explosive phenomenon due to the expanding SFS aka critical fluid upon depressurization of the biomass. In the latter case, rapid decompression is sometimes desirable. Larger systems may require longer to decompress than smaller systems. In the former case, decompression is not required to provide the desired disruption.

The nature of the biomass determines the relative importance of the two disruption mechanisms in any given application. During this first disruption step, an extract fraction may optionally be collected from the critical fluid contacting the biomass. In the second step of the fractionation, the disrupted biomass is subjected to a multiplicity of critical fluid extraction steps, the steps being characterized in that different solvation conditions are used in each. Thus, fractionation of the biomass is effected. As mentioned, critical fluid solvation properties may be varied by adjusting pressure, temperature, or modifier concentration. These parameters may be adjusted individually or in combination. Solvation conditions may also be varied through the use of different modifiers in a single fractionation procedure, although this would not typically be advantageous.

Preferably, each subsequent critical fluid is altered to change the solvation properties of the extracting fluid, so that each step can recover a different spectrum of compounds. The solvation properties of critical fluids can be altered by changing the temperature or pressure of the fluid. By way of example, a preferred temperature and pressure for a critical fluid comprising carbon dioxide is a temperature in the range of 10° C. to 60° C. and a pressure in the range of 500 psig to 5,000 psig.

Preferred critical fluids comprise carbon dioxide, nitrous oxide, ethylene, ethane, propane and freons. The fluid may also contain modifiers. Preferred modifiers are methanol, ethanol, propanol, butanol, methylene chloride, ethyl acetate and acetone.

A preferred modifier includes methanol. In one preferred embodiment, each subsequent extraction employs a larger concentration of methanol. Thus, the plurality of critical fluids becomes increasingly more hydrophilic. The first extraction step tends to remove lipophilic compounds while the last extraction step tends to remove hydrophilic compounds. Removal of the lipophilic materials allows the next more hydrophilic critical fluid to have access to more hydrophilic compounds trapped in cellular structures. Preferred methanol concentration ranges for a first extraction step on disrupted biomass, based on carbon dioxide at a pressure of 3000 psig and a temperature of 40° C., are 0-5 volume %. For the same temperature and pressure, 5-10 volume % methanol is preferred for a second extraction step; 10-20 volume % methanol is preferred for a third extraction step; 20-30 volume % methanol is preferred for a fourth extraction step; 30-50 volume % methanol is preferred for a fifth extraction step.

The combination of disruption and extraction with critical fluids produces larger numbers of fractions exhibiting biological activity than corresponding fractions derived from conventional organic solvent extractions. The use of critical fluids allows for easy removal of much of the solvent by mere depressurization. Use of a single apparatus to perform both the disruption and extraction steps minimizes labor and increases efficiency. Indeed, the entire process can be readily automated. The use of critical fluids allows the extraction conditions to be readily varied by temperature, pressure, or modifier solvents. Use of critical fluids for both the disruption and extraction simplifies the procedure and minimizes equipment needs, processing time, potential for contamination, and loss of yield. These and other features and advantages will be readily apparent from the drawing and detailed discussion which follow.

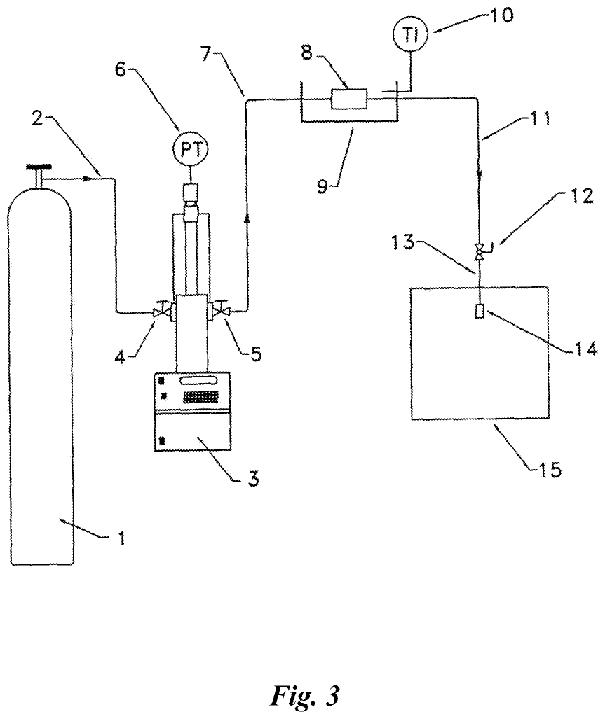

An alternative embodiment for lipid extraction is shown in FIG. 3. SuperFluids aka critical fluid extractions were carried out on an ISCO (Lincoln, Nebr.) SFX 3560 automated extractor. As shown in FIG. 3, this is a dual pump system, utilizing syringe pump 1 for neat critical fluid and syringe pump 2 for modifier. The pumps are independently controllable, allowing easy adjustment of the fluid composition. To prepare a sample, the culture was centrifuged at 8000 g for 10 min. The cell pellet was collected after decanting the supernatant, transferred to a polystyrene weighing dish, and dried at 25° C.-37° C. for 1 day, with or without vacuum. The dried cell pellet was transferred to a 10 ml ISCO extraction cartridge, numbered 3 in FIG. 3, after which the cartridge was filled with 3 mm diameter glass beads to reduce the dead volume.

After loading a cartridge on the cartridge holder, the disruption/extraction procedure was commenced. The system was brought to 3000 psig and 40° C., and extracted for 10 minutes with pure CO₂. This fraction was collected in methanol in a glass vial, numbered 4 in FIG. 3. Next, depressurization was carried out in a period of less than about 5 minutes in order to disrupt the cells. Next, the extraction parameters were set to: Supercritical CO₂ at 3000 psig and extraction temperature 40 .degrees C., step extractions with methanol as cosolvent at 0, 5, 10, 20, and 50 vol % (the modifier content of the last fraction was varied as described below), each step being 10 min. Because some void volume remained between the glass beads, the composition of the extraction medium did not change sharply or immediately when modifier flowrate was adjusted to give a new fluid composition. Each sample thus yielded 6 fractions, which were collected in methanol in separate glass vials. The different collection vials are mounted in a carousel, numbered 5 in the figure. The vials are automatically positioned by the SFX 3560 extractor apparatus. While the preceding steps were carried out in a continuous flow mode, cessation of flow to allow static contact time is also contemplated. This procedure may allow a reduction in the amount of extraction solvent required.

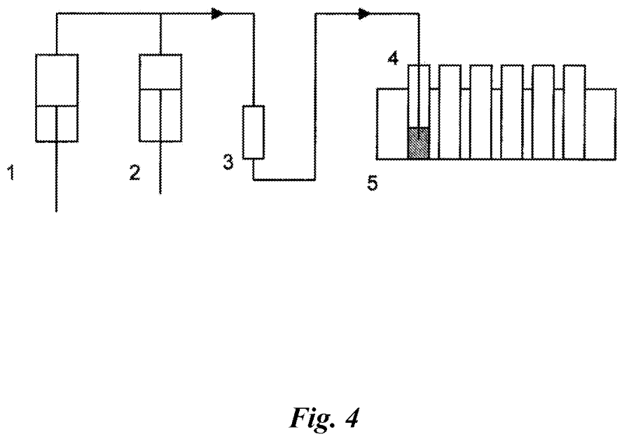

With reference to FIG. 4, the apparatus for practicing the process of FIG. 1 is shown. The desired amount of solid yeast powder is loaded into contact chamber 8. The chamber is sealed and connected to the system between inlet line 7 and outlet line 11. To allow temperature control, the chamber 8 is immersed in temperature bath 9, instrumented with temperature indicator 10.

Critical fluid contained in cylinder 1 is supplied through line 2 and valve 4 to high pressure pump 3. With valve 12 closed and valve 5 open, high pressure pump 3 pressurizes line 7, chamber 8, and line 11. Pressure is indicated by pressure transducer 6. Once chamber 8 has been pressurized, the yeast cell wall and critical fluid are allowed a certain amount of contact time. After the desired contact time, valve 12 is quickly opened, e.g., in less than about 1 second, causing rapid depressurization of critical fluid with entrained yeast cell wall into the depressurization receptacle 15.

Depressurization may be carried out through a nozzle device 14, of which many designs are available. Some nozzle designs include impingement surfaces that increase mechanical shear by deflecting the discharging material.

The depressurization receptacle 15 is substantially larger than the contact chamber and operates at only a low pressure. It may be open to the atmosphere via a filter, which would trap any potentially escaping particles, although this is not shown in the figure. Alternatively, depressurization receptacle 15 may be a flexible container such as a plastic bag. After depressurization, yeast cell wall nanoparticles are collected from the depressurization receptacle 15 for analysis.

The general operation of the equipment was as described in the explanation of FIG. 1. The syringe pump 3 was filled with CO₂, propane, Freon 22 or N₂ and compressed to the operating pressure. The yeast cell wall was added to the contact chamber 8 (volume 11 mL), which was then connected to the outlet tube 11. The letdown ball valve 12 was shut. The pump was started at a constant pressure, which was determined for each particular run. The pump outlet valve 5 was opened and the critical fluid allowed to pressurize the system. The yeast cell wall was contacted with critical fluid for a predetermined time, with the contact chamber 8 submerged in an acetone/dry ice, liquid nitrogen, or warm water bath 9 to control temperature. The pump outlet valve 5 was shut and then the letdown valve 12 was opened to decompress the contents of the unit in less than about 1 second into a depressurization bag. The samples were blown out through a 0.120 inch inside diameter nozzle. The samples were collected from the bag and viewed under a microscope to determine size.

It is intended that the subject matter contained in the preceding description be intended in an illustrative rather than a limiting sense.

What is claimed is:

1. A method for fractionating yeast cells, including the steps of:
contacting yeast cells with SuperFhuids™ under pressure,
rapidly releasing the saturated yeast slurry into a decompression chamber to release intracellular proteins, enzymes and nucleic acids;
spray-drying the disrupted yeast cells by heating during decompression into a partially or fully evacuated decompression chamber;
re-contacting the spray dried disrupted yeast cells with SuperFluids™ to extract lipids;
rapid expansion of SuperFluids™ saturated spray-dried-disrupted yeast cells to produce yeast cell wall nanoparticles; and
subjecting the nanoparticles enzymatic cleavage of yeast cell nanoparticles to produce Beta-glucans, chitins and mannans fractions.

2. The method of claim 1, further including hydrolysis of yeast cell nanoparticles to produce Beta-glucans, chitins and mannans fractions.

3. The method of claim 2, wherein the hydrolysis is an acid-based hydrolysis.

4. The method of claim 1, wherein during decompression, the yeast solution is heated so the liquid solvent (water) evaporates and the disrupted yeast is dried into a powder as in a spray drier.

5. The method of claim 1, wherein yeast is decompressed into a fully or partially evacuated chamber to achieve a spray drying effect.

6. The method of claim 1, wherein a combination of heat and low pressure can be utilized to produce a spray-dried disrupted yeast powder.

7. The method of claim 1, wherein SuperFluids at appropriate conditions of temperature and pressure is used to extract and remove lipids from the spay-dried yeast powder; and wherein
the spray dried disrupted yeast powder is contacted with SuperFluids at operating pressures between 500 and 5,000 prig and temperatures between 10° C. and 100° C. to solubilize and remove lipids.

8. The method of claim 1, wherein the lipid-reduced, disrupted, spray-dried yeast powder saturated with SuperFluids is rapidly expanded to produce yeast wall nanoparticles.

9. The method of claim 1, wherein the lipid-reduced, disrupted, spray-dried yeast nanoparticles are processed by enzymatic cleavage to produce fractions of β-glucans, chitins and mannans.

10. The method of claim 1, wherein, the lipid-reduced, disrupted, spray-dried yeast nanoparticles are processed by acid hydrolysis to produce fractions of β-glucans, chitins and mannans.

11. The method of claim 1, wherein, the lipid-reduced, disrupted, spray-dried yeast nanoparticles are processed by a combination of acid hydrolysis and enzymatic cleavage to produce fractions of β-glucans, chitins and mannans.

12. A product produced by contacting yeast with a SuperFluids under pressure, including the steps of:
rapidly releasing the saturated yeast slurry into a decompression chamber to release intracellular proteins, enzymes and nucleic acids;
spray drying the disrupted yeast cells by heating during decompression and/or decompression into a partially or fully evacuated decompression chamber;
re-contacting the spray dried disrupted yeast cells with SuperFluids to extract lipids;
rapid expansion of SuperFluids saturated spray-dried-disrupted yeast cells to produce yeast cell wall nanoparticles;
enzymatic cleavage of yeast cell nanoparticles to product Beta-glucans, chitins and mannans fractions, or;
hydrolysis of yeast cell nanoparticles to product Beta-glucans, chitins and mannans fractions, or;
enzymatic cleavage and hydrolysis of yeast cell nanoparticles to product Beta-glucans, chitins and mannans fractions.

13. The product of claim 12, wherein the enzymatic cleavage and hydrolysis of yeast cell nanoparticles to product Beta-glucans, chitins and mannans fractions.

14. The product of claim 12, wherein the hydrolysis is an acid-based hydrolysis.

15. The product of claim 12, wherein during decompression, the yeast solution is heated so the liquid solvent (water) evaporates and the disrupted yeast is dried into a powder as in a spray drier and wherein yeast is decompressed into a fully or partially evacuated chamber to achieve a spray drying effect.

16. The product of claim 12, wherein SuperFluids™ CO₂ at appropriate conditions of temperature and pressure is used to extract and remove lipids from the spay-dried yeast powder; and wherein
the spray dried disrupted yeast powder is contacted with SuperFluids C0₂ at operating pressures between 2,000 and 20,000 psig and temperatures between 20 and 100° C. to solubilize and remove lipids.

17. The product of claim 12, wherein the lipid-reduced, disrupted, spray-dried yeast nanoparticles are processed by enzymatic cleavage to produce fractions of β-glucans, chitins and mannans.

18. The product of claim 12, wherein, the lipid-reduced, disrupted, spray-dried yeast nanoparticles are processed by hydrolysis to produce fractions of β-glucans, chitins and mannans.

19. The product of claim 12, wherein, the lipid-reduced, disrupted, spray-dried yeast nanoparticles are processed by a combination of acid hydrolysis and enzymatic cleavage to produce fractions of β-glucans, chitins and mannans.

20. A product derived from the fractionation of yeast, wherein the yeast is subjected to critical, supercritical, or near critical fluids for disruption of yeast cells.
